(19) United States
(12) Patent Application Publication
(10) Pub. No.: US 2009/0042839 A1
(43) Pub. Date: Feb. 12, 2009

(54) Crystalline forms of ibandronate sodium

(76) Inventors: Sharon Avhar-Maydan, Givataym (IL); Claude Singer, Kfar Saba (IL); Tamas Koltai, Netanya (IL); Alexandr Jegorov, Dobra Voda (CZ)

Correspondence Address: KENYON & KENYON LLP, ONE BROADWAY, NEW YORK, NY, 10004, US

(21) Appl. No.: 12/152,830

(22) Filed: May 15, 2008

Related U.S. Application Data
(60) Provisional application No. 60/954,959, filed on Aug. 9, 2007.
(60) Provisional application No. 60/985,837, filed on Nov. 6, 2007.
(60) Provisional application No. 61/003,353, filed on Nov. 16, 2007.

(51) Int. Cl. A61K 31/663; C07F 9/28; A61P 19/00

(52) U.S. Cl. 514/108; 568/14

(57) ABSTRACT

Provided are crystalline forms of ibandronate sodium, as well as processes for the preparation thereof.

### CROSS-REFERENCE TO RELATED APPLICATIONS

This application claims the benefit of U.S. provisional application Ser. Nos. 60/954,959, filed Aug. 9, 2007; 60/985,837, filed Nov. 6, 2007; and 61/003,353 filed Nov. 16, 2007, each of which is hereby incorporated by reference in its entirety.

### FIELD OF THE INVENTION

The invention encompasses crystalline forms of ibandronate sodium, as well as processes for the preparation thereof.

### BACKGROUND OF THE INVENTION

Ibandronate sodium, (1-hydroxy-3-(N-methyl-N-pentylamino)propylidene) bisphosphonic acid monosodium salt, is a third-generation nitrogen-containing bisphosphonate characterized by an aliphatic tertiary amine side chain. Ibandronate sodium is typically a white powder. Ibandronate sodium has the empirical formula C₉H₂₂NO₇P₂Na and the following chemical structure.

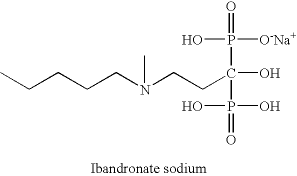

Ibandronate sodium is currently marketed in the United States by Hoffmann-La Roche under the tradename BONIVA® in its monohydrate form. BONIVA® is indicated for the treatment and prevention of osteoporosis in post-menopausal women. BONIVA® is available as an intravenous injection administered every 2-3 months or as an oral formulation. BONIVA® is marketed in Europe under the tradename BONDRONAT® for the treatment of skeletal-related events in patients with breast cancer and bone metastases. BONDRONAT® is available in an ampoule with 1 ml concentrate for solution for infusion; 1 ml of solution is reported to contain 1.125 mg of ibandronic monosodium salt monohydrate, corresponding to 1 mg of ibandronic acid.

Ibandronate salts, such as ibandronate sodium, are generally prepared from ibandronic acid (“IBD-Ac”), which has the following chemical structure:

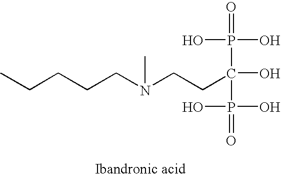

U.S. Pat. No. 4,927,814 discloses diphosphonic acids, such as ibandronic acid, derivatives thereof, processes for preparing the acids and derivatives, and pharmaceutical compositions containing them.

The invention relates to the solid state physical properties of ibandronate sodium. These properties can be influenced by controlling the conditions under which ibandronate sodium is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must necessitate the use of glidants such as colloidal silicon dioxide, talc, starch, or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally administered active ingredient can reach the patient's bloodstream. The rate of dissolution is also a consideration in formulation syrups, elixirs, and other liquid medicaments. The solid state form of a compound can also affect its behavior on compaction and its storage stability.

These practical physical characteristics are influenced by the conformation and orientation of molecules in the unit cell, which define a particular polymorphic form of a substance. The polymorphic form can give rise to thermal behavior different from that of the amorphous material or another polymorphic form. Thermal behavior is measured in the laboratory by such techniques as capillary melting point, thermogravimetric analysis (“TGA”), and differential scanning calorimetry (“DSC”) and can be used to distinguish some polymorphic forms from others. A particular polymorphic form can also give rise to distinct spectroscopic properties that can be detectable by powder x-ray crystallography, solid state ¹³C NMR spectroscopy, and infrared spectrometry.

Generally, a crystalline solid has improved chemical and physical stability over the amorphous form, and forms with low crystallinity. Crystalline forms may also exhibit improved solubility, hygroscopicity, bulk properties, and/or flowability.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

PCT Publication No. WO 2006/024024 refers to several crystalline forms of ibandronate sodium and processes for their preparation.

There is a need in the art for additional polymorphic forms of ibandronate sodium.

### SUMMARY OF THE INVENTION

In one embodiment, the invention encompasses a crystalline form of ibandronate sodium denominated Form C2. Form C2 is characterized by x-ray powder diffraction reflections at 10.7, 13.3, 18.0, and 20.4°2θ±0.2°2θ. The crystalline ibandronate sodium Form C2 may be prepared by a process comprising suspending ibandronic acid Form S1 with n-butanol; heating the slurry; adding a solution of sodium methoxide in methanol to the slurry to obtain ibandronate sodium; and cooling the slurry to obtain a precipitate of the crystalline ibandronate sodium Form C2. The crystalline ibandronate sodium Form C2 may be also prepared by a process comprising slurrying ibandronate sodium Form V in ethanol to obtain a precipitate.

In another embodiment, the invention encompasses a crystalline form of ibandronate sodium denominated Form Alpha. Form Alpha is characterized by x-ray powder diffraction reflections at 11.8, 16.2, 17.8, and 18.8°2θ±0.2°2θ. The crystalline ibandronate sodium Form Alpha may be prepared by a process comprising suspending ibandronic acid Form S1 with a solvent; heating the suspension; adding a sodium salt to the suspension to obtain ibandronate sodium; and cooling the suspension to obtain a precipitate of the ibandronate sodium Form Alpha, wherein the solvent is selected from the group consisting of ethanol, n-butanol, and 2-butanol; and the sodium salt is selected from the group consisting of sodium carbonate anhydrous, sodium citrate dihydrate and sodium hydrogen carbonate.

In yet another embodiment, the invention encompasses a crystalline form of ibandronate sodium denominated Form S4. Form S4 is characterized by x-ray powder diffraction reflections at 8.7, 11.3, 13.0, and 13.5°2θ±0.2°2θ. The crystalline ibandronate sodium Form S4 may be prepared by a process comprising suspending ibandronic acid Form S1 with 1-propanol; heating the suspension; adding sodium hydrogen carbonate or sodium carbonate to the suspension to obtain ibandronate sodium; and cooling the suspension to obtain a precipitate of the crystalline ibandronate sodium Form S4.

In one embodiment, the invention encompasses a crystalline form of ibandronate sodium denominated Form S5. Form S5 is characterized by x-ray powder diffraction reflections at 8.9, 10.4, 12.0, and 16.3°2θ±0.2°2θ. The crystalline ibandronate sodium Form S5 may be prepared by a process comprising suspending ibandronic acid Form S1 with 2-propanol; heating the suspension; adding a base selected from the groups consisting of: sodium carbonate anhydrous, sodium bicarbonate, sodium citrate and sodium methoxide to the suspension to obtain ibandronate sodium; and cooling the suspension to obtain a precipitate of ibandronate sodium Form S5.

In another embodiment, the invention encompasses a crystalline form of ibandronate sodium denominated Form Beta. Form Beta is characterized by x-ray powder diffraction reflections at 12.3, 17.3, 18.5, and 19.0°2θ±0.2°2θ. The crystalline ibandronate sodium Form Beta may be prepared by a process comprising slurrying ibandronate di-sodium in 1-propanol; heating; and cooling to obtain a precipitate.

In another embodiment, the invention encompasses a process for preparing a mixture of crystalline ibandronate sodium Form C and Form Alpha comprising suspending ibandronic acid Form S1 with n-butanol; heating the suspension; adding a sodium salt to the suspension; and cooling the suspension to obtain a precipitate of the mixture of crystalline ibandronate sodium Form C and Form Alpha, wherein the sodium salt is selected from the group consisting of sodium tert butoxide and sodium acetate anhydrous.

In another embodiment, the invention encompasses crystalline ibandronate sodium Forms C2, Alpha, S4, S5, or Beta as defined in any of the embodiments mentioned herein having a maximal particle size of less than about 500 μm, more preferably less than about 300 μm, even more preferably less than about 200 μm, even more preferably less than about 100 μm, and most preferably less than about 50 μm.

The invention also encompasses a crystalline form of ibandronate sodium denominated Form n-butanol solvate. Form n-butanol solvate is characterized by x-ray powder diffraction reflections at 9.2, 13.9, 16.6, and 18.5°±0.2°2θ. The crystalline ibandronate sodium Form n-butanol solvate may be prepared by a process comprising dissolving ibandronate sodium in water; combining the solution with n-butanol; and removing the water and n-butanol to obtain a precipitate of the crystalline ibandronate sodium Form n-butanol solvate.

The invention also encompasses a crystalline form of ibandronate sodium denominated Form i-butanol solvate. The crystalline ibandronate sodium Form i-butanol solvate may be prepared by a process comprising dissolving ibandronate sodium in water; combining the solution with i-butanol; and removing the water and i-butanol to obtain a precipitate of the crystalline ibandronate sodium Form i-butanol solvate.

The invention also encompasses a crystalline form of ibandronate sodium denominated Form pyridine solvate. Form pyridine solvate is characterized by x-ray powder diffraction reflections at 10.6, 15.1, 15.9, and 17.8°2θ±0.2°2θ. The crystalline ibandronate sodium Form pyridine solvate may be prepared by a process comprising dissolving ibandronate sodium in water; and adding pyridine to the solution to obtain a precipitate of the crystalline ibandronate sodium Form pyridine solvate.

In yet another embodiment, the invention encompasses a pharmaceutical formulation comprising at least one of the above-described crystalline ibandronate sodium Forms C2, Alpha, S4, S5, Beta, i-butanol solvate, n-butanol solvate or pyridine solvate, and at least one pharmaceutically acceptable excipient.

In one embodiment, the invention encompasses a process for preparing a pharmaceutical formulation comprising combining at least one of the above-described crystalline ibandronate sodium Forms C2, Alpha, S4, S5, Beta, i-butanol solvate, n-butanol solvate or pyridine solvate, with at least one pharmaceutically acceptable excipient.

In one embodiment, the invention encompasses the use of the above-described crystalline ibandronate sodium Forms C2, Alpha, S4, S5, Beta, i-butanol solvate, n-butanol solvate or pyridine solvate, in the manufacture of a pharmaceutical composition.

In another embodiment, the invention encompasses at least one of the above-described crystalline ibandronate sodium Forms C2, Alpha, S4, S5, Beta, i-butanol solvate, n-butanol solvate or pyridine solvate, for use in treating or preventing skeletal-related events, preferably wherein the skeletal-related event is osteoporosis.

### BRIEF DESCRIPTION OF THE DRAWINGS

FIG. 1 illustrates a characteristic x-ray powder diffractogram of crystalline ibandronate sodium Form C2.

FIG. 2 illustrates a characteristic x-ray powder diffractogram of crystalline ibandronate sodium Form Alpha prepared according to example 1.

FIG. 3 illustrates a characteristic x-ray powder diffractogram of crystalline ibandronate sodium Form Alpha prepared according to example 3.

FIG. 4 illustrates a characteristic x-ray powder diffractogram of crystalline ibandronate sodium Form S4.

FIG. 5 illustrates a characteristic x-ray powder diffractogram of crystalline ibandronate sodium Form S5.

FIG. 6 illustrates a characteristic x-ray powder diffractogram of crystalline ibandronate sodium Form Beta.

FIG. 7 illustrates a characteristic x-ray powder diffractogram of crystalline ibandronate sodium Form C2 prepared according to example 13.

FIG. 8 illustrates a characteristic x-ray powder diffractogram of crystalline ibandronate sodium Form n-butanol solvate.

FIG. 9 illustrates a characteristic x-ray powder diffractogram of crystalline ibandronate sodium Form i-butanol solvate.

FIG. 10 illustrates a characteristic x-ray powder diffractogram of crystalline ibandronate sodium Form pyridine solvate.

### DETAILED DESCRIPTION OF THE INVENTION

The invention addresses a need in the art by providing additional crystalline forms of ibandronate sodium, as well as processes for their preparation. The invention also provides additional processes for preparing known crystalline forms of ibandronate sodium. The present invention provides crystalline forms that

As used herein, unless otherwise defined, the term “room temperature” refers to a temperature of about 15° C. to about 30° C.

As used herein, unless otherwise defined, the term purity refers to crystalline purity. Crystalline purity may refer to the presence or absence of one or more crystalline forms other than the referenced crystalline form of the compound.

As used herein, a slurry or suspension refers to a heterogeneous mixture where complete dissolution does not occur.

The present invention relates to polymorphic forms, preferably wherein the each polymorphic form is substantially free of any other polymorphic forms (or substantially free of polymorph forms Y and Z). By “substantially free” is meant 20% or less, preferably 10% or less, more preferably 5% or less, most preferably 2% or less, particularly 1% or less, more particularly 0.5% or less and most particularly 0.2% or less as measured by PXRD.

PCT Publication No. WO 2006/024024 (“WO '024”), hereby incorporated by reference, refers to the following crystalline forms of ibandronate sodium, as well as processes for preparing them: Form C, Form D, Form E, Form F, Form G, Form H, Form J, Form K, Form K2, Form K3, Form Q, Form Q1, Form Q2, Form Q3, Form Q4, Form Q5, Form Q6, Form QQ, Form R, Form S, Form T. WO '024 reports several characteristic powder x-ray diffraction (“PXRD”) reflections for each crystalline form of ibandronate sodium. These characteristic reflections are summarized in Table 1 below.

TABLE 1
Characteristic PXRD Reflections Reported in WO ′024 for Crystalline Forms of
Ibandronate Sodium

| Crystalline | Primary Characteristic PXRD | Secondary Characteristic PXRD |
|---|---|---|
| Form | Reflections | Reflections |
| Form C | 4.7, 5.0, 17.2, 18.3 and 19.5 °2θ ± | 17.6, 19.7, 20.2, 20.6, and 23.8 °2θ ± |
| | 0.2 °2θ | 0.2 °2θ |
| Form D | 4.8, 9.3, 18.5, 23.1, and 36.1 °2θ ± | 15.3, 19.9, 26.3, 27.2, and 30.4 °2θ ± |
| | 0.2 °2θ | 0.2 °2θ |
| Form E | 4.6, 4.8, 5.3, 9.3, and 34.7 °2θ ± | 18.6, 23.3, 24.5, 27.1, and 30.1 °2θ ± |
| | 0.2 °2θ | 0.2 °2θ |
| Form F | 4.9, 5.1, 6.0, 20.0, and 36.4 °2θ ± | 18.6, 26.0, 28.5, 30.4, and 31.3 °2θ ± |
| | 0.2 °2θ | 0.2 °2θ |
| Form G | 4.7, 9.2, 17.4, 18.4, and 19.9 °2θ ± | 10.1, 15.2, 18.7, 26.3, and 27.1 °2θ ± |
| | 0.2 °2θ | 0.2 °2θ |
| Form H | 4.8, 5.7, 17.3, 19.5, and 26.0 °2θ ± | 18.5, 20.1, 23.8, 31.1, and 37.1 °2θ ± |
| | 0.2 °2θ | 0.2 °2θ |
| Form J | 4.6, 9.2, 18.3, 19.6, and 25.6 °2θ ± | 17.5, 18.9, 21.7, 22.9, and 29.5 °2θ ± |
| | 0.2 °2θ | 0.2 °2θ |
| Form K | 5.0, 5.9, 17.2, 20.0, and 25.9 °2θ ± | 18.5, 19.7, 21.4, 26.5, and 31.1 °2θ ± |
| | 0.2 °2θ | 0.2 °2θ |
| Form K2 | 5.1, 6.1, 17.3, 20.1, and 21.5 °2θ ± | 18.6, 19.6, 26.1, 26.8, and 31.1 °2θ ± |
| | 0.2 °2θ | 0.2 °2θ |
| Form K3 | 5.1, 6.2, 17.3, 19.7, and 20.1 °2θ ± | 18.5, 21.5, 23.8, 25.8, and 31.1 °2θ ± |
| | 0.2 °2θ | 0.2 °2θ |
| Form Q | 5.0, 6.1, 17.2, 25.7, and 30.9 °2θ ± | 16.8, 21.4, 26.7, 29.1, and 36.9 °2θ ± |
| | 0.2 °2θ | 0.2 °2θ |
| Form Q1 | 4.7, 6.0, 17.2, 26.2, and 31.0 °2θ ± | 19.5, 21.4, 25.8, 29.1, and 37.1 °2θ ± |
| | 0.2 °2θ | 0.2 °2θ |
| Form Q2 | 4.9, 6.2, 25.9, 31.0, and 37.1 °2θ ± | 16.9, 17.3, 19.0, 26.6, and 29.2 °2θ ± |
| | 0.2 °2θ | 0.2 °2θ |
| Form Q3 | 5.9, 17.1, 19.6, 20.2, and 21.3 °2θ ± | 18.0, 18.5, 23.6, 24.7, and 30.8 °2θ ± |
| | 0.2 °2θ | 0.2 °2θ |
| Form Q4 | 6.1, 17.2, 19.6, 20.3, and 21.4 °2θ ± | 16.9, 18.1, 18.5, 23.7, and 24.8 °2θ ± |
| | 0.2 °2θ | 0.2 °2θ |
| Form Q5 | 6.1, 17.2, 19.6, 20.1, and 21.5 °2θ ± | 16.8, 24.7, 25.7, 29.0, and 30.9 °2θ ± |
| | 0.2 °2θ | 0.2 °2θ |
| Form Q6 | 6.1, 17.3, 19.6, 21.5, and 30.8 °2θ ± | 16.9, 20.2, 25.6, 26.9, and 29.1 °2θ ± |
| | 0.2 °2θ | 0.2 °2θ |
| Form QQ | 6.2, 25.9, 26.7, 31.1, and 37.2 °2θ ± | 16.9, 17.3, 21.5, 24.7, and 29.2 °2θ ± |
| | 0.2 °2θ | 0.2 °2θ |
| Form R | 5.3, 6.0, 17.2, 18.7, and 20.0 °2θ ± | 20.5, 25.0, 26.5, 29.1, and 31.0 °2θ ± |
| | 0.2 °2θ | 0.2 °2θ |
| Form S | 4.8, 5.1, 5.3, 5.4, and 6.1 °2θ ± 0.2 | 10.5, 21.0, 26.3, 33.0, and 38.2 °2θ ± |
| | °2θ | 0.2 °2θ |
| Form T | 6.2, 15.7, 26.3, 32.6, and 35.6 °2θ ± | 17.6, 19.4, 26.9, 31.7, and 38.7 °2θ ± |
| | 0.2 °2θ | 0.2 °2θ |

As used herein, Forms C, D, E, F, G, H, J, K, K2, K3, Q, Q1, Q2, Q3, Q4, Q5, Q6, QQ, R, S, and T of ibandronate sodium are as defined in WO2006024024, and can be made by the processes disclosed therein. Thus, as used herein ibandronic acid Forms C, D, E, F, G, H, J, K, K2, K3, Q, Q1, Q2, Q3, Q4, Q5, Q6, QQ, R, S, and T are characterized by the “primary” PXRD peaks as listed in the second column of Table 1 above, and may further be characterized by the “secondary” PXRD peaks listed in the third column of Table 1 above.

The invention encompasses additional crystalline forms of ibandronate sodium, denominated Forms C2, Alpha, S4, S5, Beta, i-butanol solvate, n-butanol solvate or pyridine solvate,

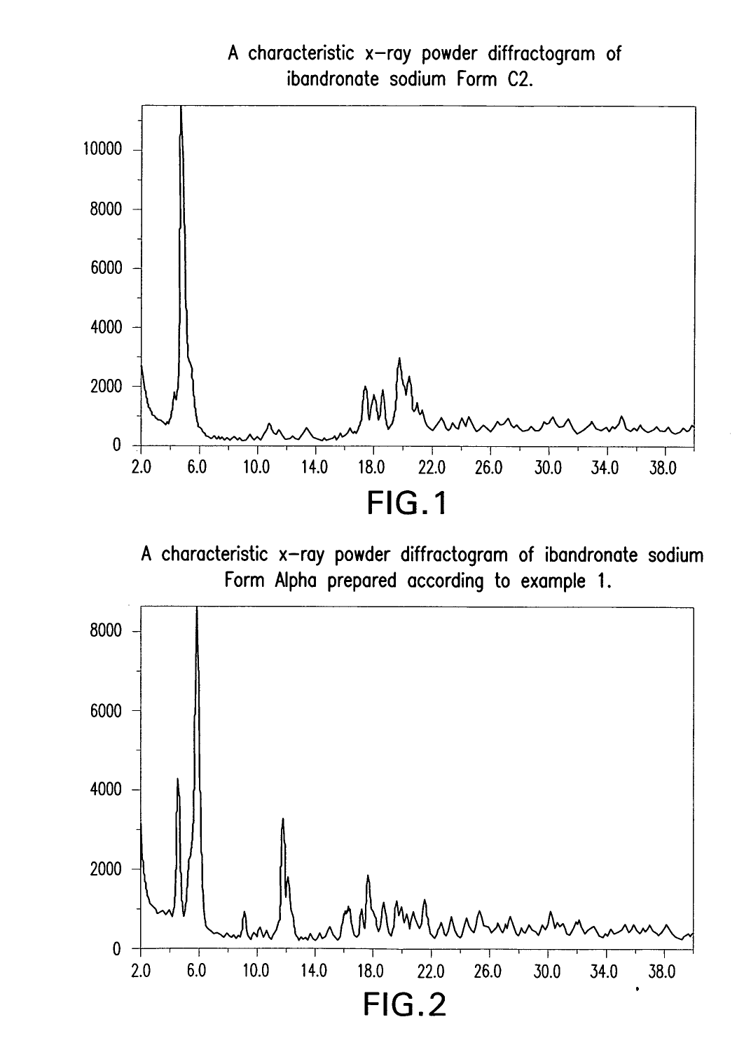

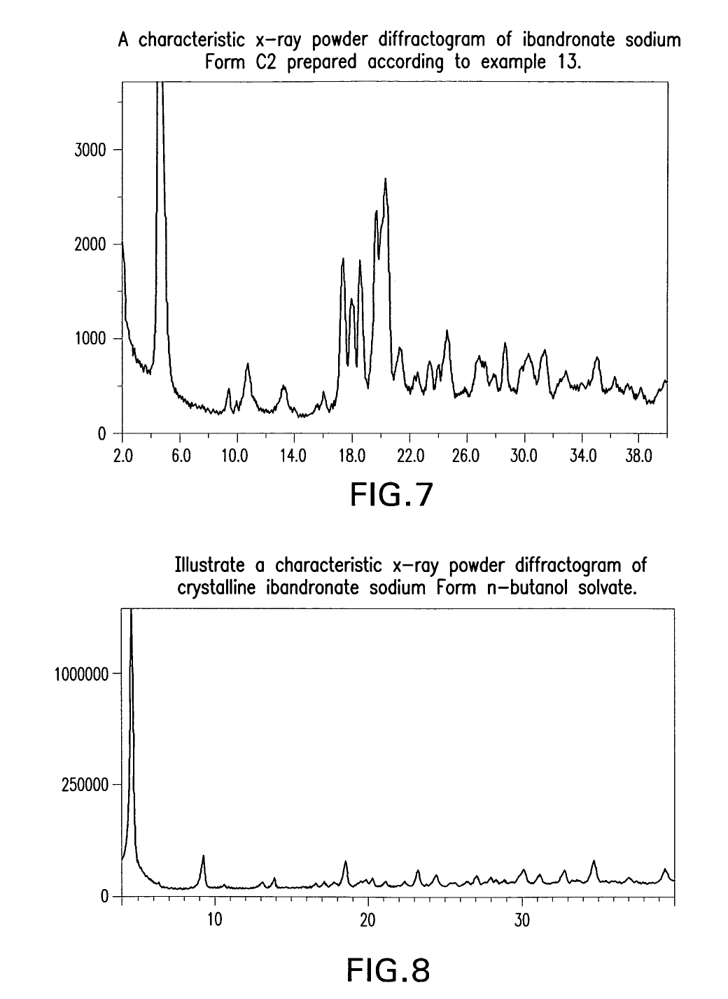

The invention encompasses a crystalline form of ibandronate sodium denominated Form C2. Form C2 is characterized by x-ray powder diffraction reflections at 10.7, 13.3, 18.0, and 20.4°2θ±0.2°2θ. Form C2 can be further characterized by x-ray powder diffraction reflections at 11.4, 17.4, 20.9, and 22.6°2θ±0.2°2θ. FIGS. 1 and 7 illustrate representative powder x-ray diffraction diagrams for Form C2. Form C2 may be a solvate of n-butanol.

One of ordinary skill in the art is aware that there is a certain amount of experimental error inherent in PXRD techniques. See, e.g., U.S. PHARMACOPEIA, 387-89 (30th ed. 2007), hereby incorporated by reference. As to individual peaks, peak positions are reported over a range of ±0.2°2θ to account for this experimental error. As to PXRD patterns in their entirety, the term “as depicted” in a particular figure is meant to account for this experimental error, as well as for variations in peak position and intensity due to factors such as, for example, variations in sample preparation, instrumentation, and the skill of the operator of the instrument. A PXRD pattern “as depicted” in a particular figure means that one of ordinary skill in the art, understanding the experimental error involved in powder X-ray diffraction techniques, would determine that the PXRD pattern corresponds to the same crystalline structure as the PXRD pattern depicted in the figure.

The crystalline ibandronate sodium Form C2 may be prepared by a process comprising suspending ibandronic acid Form S1 in n-butanol; heating the suspension; adding a solution of sodium methoxide in methanol to the suspension to obtain ibandronate sodium; and cooling the suspension to obtain a precipitate of the crystalline ibandronate sodium Form C2.

The ibandronic acid Form S1 may be prepared by the process disclosed in PCT Publication No. WO 2006/002348, hereby incorporated by reference. Ibandronic acid Form S1 is typically characterized by x-ray powder diffraction reflections at 8.2, 11.5, 11.9, 13.9, 18.6 and 22.2°2θ±0.2°2θ.

Prior to the heating step, the suspension is typically stirred.

Preferably, the suspension is heated to a temperature of about 65° C. to about 120° C., and more preferably to about 114° C. The suspension containing the sodium methoxide is further heated. The suspension containing the sodium methoxide is typically stirred while heating. Preferably, the suspension is stirred while heating for about 1 hour to about 5 hours.

Preferably, the suspension (slurry) is cooled to about 30° C. to about 5° C., and more preferably to about room temperature. The suspension is typically stirred while cooling. Preferably, the suspension is stirred while cooling for about 10 hours to about 50 hours, more preferably for about 24 hours to about 50 hours, even more preferably for about 40 hours to about 50 hours, and most preferably for about 48 hours.

The precipitated crystalline ibandronate sodium Form C2 may be recovered from the suspension by any method known to one of ordinary skill in the art. Preferably, the crystalline ibandronate sodium Form C2 is recovered by collecting the precipitate of crystalline ibandronate sodium Form C2 from the suspension by filtration, washing the precipitate, and drying the precipitate. Preferably, the precipitate is washed with n-butanol or methanol. Preferably, the precipitate is dried under vacuum with heating, preferably at a temperature of about 30° C. to about 60° C., more preferably at about 50° C. Preferably, the drying is done under a pressure of about 10-200 mm Hg. The precipitate may be dried for about 19 hours to about 20 hours.

The crystalline ibandronate sodium Form C2 may be also prepared by a process comprising slurrying ibandronate sodium Form V in ethanol to obtain a precipitate of the crystalline ibandronate sodium Form C2.

Crystalline ibandronate sodium Form V is characterized by x-ray powder diffraction reflections at 5.3, 17.2, 17.8 and 18.4°2θ±0.2°2 θ. Form V can be further characterized by x-ray powder diffraction reflections at 19.6 and 21.2°2θ±0.2°2θ. Processes for preparing crystalline ibandronate sodium Form V are disclosed in co-pending U.S. application Ser. No. 11/985,857, filed Nov. 16, 2007 and entitled “Crystalline Forms of Ibandronate Sodium” [Attorney docket no. 1662/A401US1], hereby incorporated by reference. The crystalline ibandronate sodium Form V may be prepared, for example, by a process comprising storing ibandronate sodium Form C at about 20° C. to about 30° C. for more than about 3 months.

Preferably, the slurry is stirred for a period of time sufficient to obtain the crystalline ibandronate sodium Form C2. Preferably, the slurry is stirred for about 20 hours to about 30 hours, and more preferably for about 24 hours. Preferably, the stirring is at about room temperature.

The precipitated crystalline ibandronate sodium Form C2 may be recovered from the slurry by any method known to one of ordinary skill in the art. Preferably, the crystalline ibandronate sodium Form C2 is recovered by collecting the precipitate of crystalline ibandronate sodium Form C2 from the slurry by filtration, washing the precipitate, and drying the precipitate. Preferably, the precipitate is washed with the same solvent used in the process. Preferably, the precipitate is dried under vacuum with heating, preferably at a temperature of about 30° C. to about 60° C., and more preferably at about 50° C. Preferably, the drying is done under a pressure of about 20 to about 30 mbar. The precipitate may be dried for about 19 hours to about 25 hours.

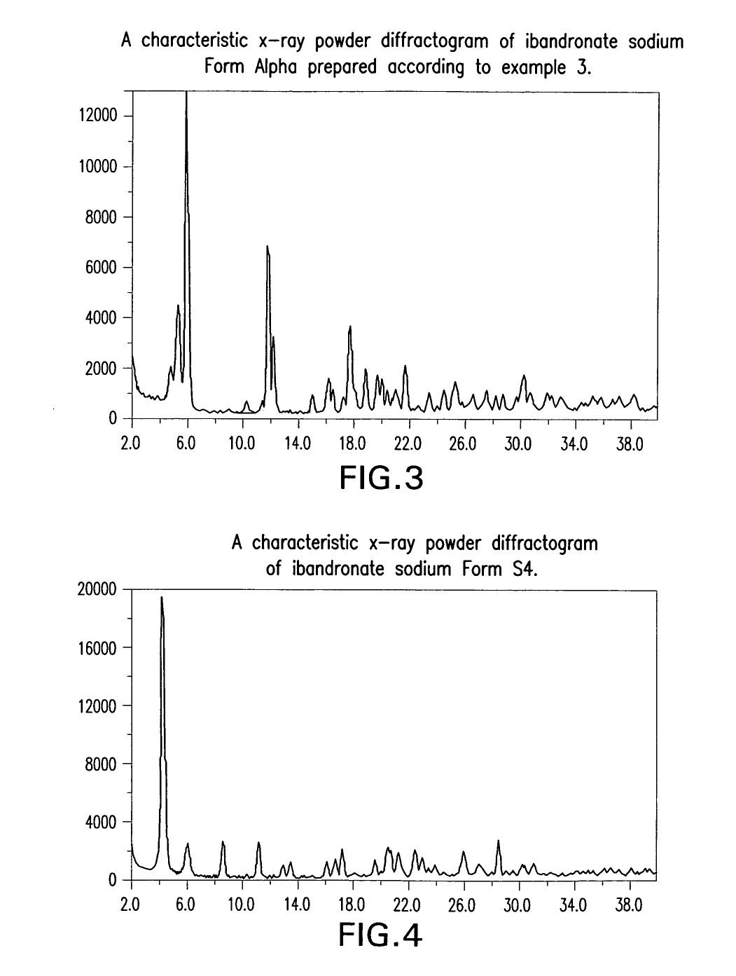

The invention also encompasses a crystalline form of ibandronate sodium denominated Form Alpha. Form Alpha is characterized by x-ray powder diffraction reflections at 11.8, 16.2, 17.8, and 18.8°2θ±0.2°2θ. Form Alpha can be further characterized by x-ray powder diffraction reflections at 10.2 and 20.0°2θ±0.2°2θ. FIGS. 2 and 3 illustrate representative powder x-ray diffraction diagrams for Form Alpha. Form Alpha may be a monohydrate.

The crystalline ibandronate sodium Form Alpha may be prepared by a process comprising suspending ibandronic acid Form S1 in a solvent; heating the suspension; adding a sodium salt to the suspension to obtain ibandronate sodium; and cooling the suspension to obtain a precipitate of the ibandronate sodium Form Alpha, wherein the solvent is selected from the group consisting of ethanol preferably absolute ethanol (water content less than 2% by volume)), n-butanol, and 2-butanol; and the sodium salt is selected from the group consisting of sodium carbonate anhydrous, sodium citrate dihydrate and sodium hydrogen carbonate.

Prior to the heating step, the suspension is typically stirred.

Preferably, the suspension is heated to a temperature of about 78° C. to about 114° C. The suspension is typically stirred while heating. Preferably, the suspension is stirring while heating for about 1 hour to about 5 hours prior to the cooling step.

Preferably, the suspension is cooled to about a temperature of about 30° C. to about 10° C., and more preferably to about room temperature. The suspension is typically stirred while cooling. Preferably, the suspension is stirred while cooling for about 16 hours to about 48 hours.

The precipitated crystalline ibandronate sodium Form Alpha may be recovered from the suspension by any method known to one of ordinary skill in the art. Preferably, the crystalline ibandronate sodium Form Alpha is recovered by collecting the precipitate of crystalline ibandronate sodium Form Alpha from the suspension by filtration, washing the precipitate, and drying the precipitate. Preferably, the precipitate is washed with the solvent used in the suspension. Preferably, the precipitate is dried under vacuum with heating, preferably at a temperature of about 30° C. to about 60° C., and more preferably at about 50° C. Preferably, the drying is done under a pressure of about 10-200 mmHg. The precipitate may be dried for about 19 hours to about 20 hours.

The invention also encompasses a crystalline form of ibandronate sodium denominated Form S4. Form S4 is characterized by x-ray powder diffraction reflections at 8.7, 11.3, 13.0, and 13.5°2θ±0.2°2θ. Form S4 can be further characterized by x-ray powder diffraction reflections at 16.2, 16.8 and 22.6°2θ±0.2°2θ. FIG. 4 illustrates a representative powder x-ray diffraction diagram for Form S4. Form S4 may be a sesquihydrate hemipropanolate

The crystalline ibandronate sodium Form S4 may be prepared by a process comprising suspending ibandronic acid Form S1 in 1-propanol; heating the suspension; adding sodium hydrogen carbonate or sodium carbonate to the suspension to obtain ibandronate sodium; and cooling the suspension (slurry) to obtain a precipitate of the crystalline ibandronate sodium Form S4.

Prior to the heating step, the suspension is typically stirred.

Preferably, the suspension is heated to a temperature of about 75° C. to about 95° C., and more preferably to about 95° C. The suspension is typically stirred while heating. Preferably, the suspension is stirred while heating for about 1 hour to about 5 hours prior to the cooling step. Preferably, the suspension is stirred at a temperature of about 70° C. to about 95° C., and more preferably at about 81° C.

Preferably, the suspension is cooled to about a temperature of about 30° C. to about 10° C., and more preferably to about room temperature. The suspension is typically stirred while cooling. Preferably, the suspension is stirred while cooling for about 10 hours to about 25 hours, more preferably, to about 16 hours.

The precipitated crystalline ibandronate sodium Form S4 may be recovered from the suspension by any method known to one of ordinary skill in the art. Preferably, the crystalline ibandronate sodium Form S4 is recovered by collecting the precipitate of crystalline ibandronate sodium Form S4 from the suspension by filtration, washing the precipitate, and drying the precipitate. Preferably, the precipitate is washed with 1-propanol. Preferably, the precipitate is dried under vacuum with heating, preferably at a temperature of about 70° C. to about 30° C., more preferably about 40° C. to about 55° C., at about 50° C. Preferably, the drying is done under a pressure of about 10-200 mm Hg. The precipitate may be dried for about 19 hours to about 20 hours.

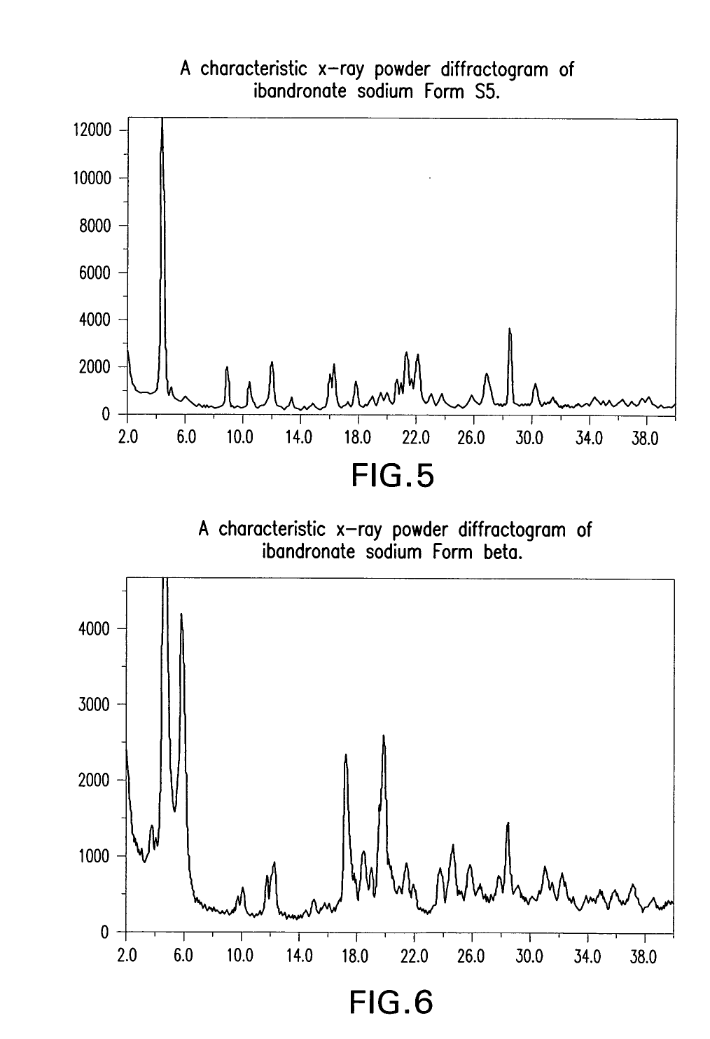

The invention also encompasses a crystalline form of ibandronate sodium denominated Form S5. Form S5 is characterized by x-ray powder diffraction reflections at 8.9, 10.4, 12.0, and 16.3°2θ±0.2°2θ. Form S5 can be further characterized by x-ray powder diffraction reflections at 16.0, 17.8 and 22.1°2θ±0.2°2θ. FIG. 5 illustrates a representative powder x-ray diffraction diagram for Form S5. Form S5 may be a monohydrate hemiisopropanolate.

The crystalline ibandronate sodium Form S5 may be prepared by a process comprising suspending (slurry) ibandronic acid Form S1 in 2-propanol; heating the suspension; adding a base selected from the group consisting of: sodium carbonate anhydrous, sodium bicarbonate, preferably sodium citrate dehydrate or anhydrous, and sodium methoxide to the suspension to obtain ibandronate sodium; and cooling the suspension to obtain a precipitate of ibandronate sodium Form S5.

Prior to the heating step, the suspension is typically stirred. Preferably, the suspension is heated to a temperature of about 60° C. to about 81° C., and more preferably at about 81° C. The suspension is typically stirred while heating. Preferably, the suspension is stirred while heating for about 1 hour to about 5 hours prior to the cooling step.

Preferably, the suspension is cooled to a temperature of about 30° C. to about 10° C., and more preferably to about room temperature. The suspension is typically stirred while cooling. Preferably, the suspension is stirred while cooling for about 10 hours to about 24 hours, and more preferably for about 16 hours.

The precipitated crystalline ibandronate sodium Form S5 may be recovered from the suspension by any method known to one of ordinary skill in the art. Preferably, the crystalline ibandronate sodium Form S5 is recovered by collecting the precipitate of crystalline ibandronate sodium Form S5 from the suspension by filtration, washing the precipitate, and drying the precipitate. Preferably, the precipitate is washed with 2-propanol. Preferably, the precipitate is dried under vacuum with heating, preferably at a temperature of about 40° C. to about 60° C., and more preferably at a temperature of about 50° C. Preferably, the drying is done under a pressure of about 10-200 mm Hg. The precipitate may be dried for about 10 hours to about 48 hours, and preferably for about 24 hours.

The invention also encompasses a crystalline form of ibandronate sodium denominated Form Beta. Form Beta is characterized by x-ray powder diffraction reflections at 12.3, 17.3, 18.5, and 19.0°2θ±0.2°2θ. Form Beta can be further characterized by x-ray powder diffraction reflections at 22.0, 23.8 and 24.7°2θ±0.2°2θ. FIG. 6 illustrates a representative powder x-ray diffraction diagram for Form Beta. Form beta may be a monobutanolate.

The crystalline ibandronate sodium Form Beta may be prepared by a process comprising slurrying ibandronate di-sodium and ibandronic acid in 1-propanol; heating the slurry; and cooling the slurry to obtain a precipitate of crystalline ibandronate sodium Form Beta.

Preferably, the slurry is stirred during the process. Preferably, the slurry is stirred for about 18 hours to about 30 hours, and more preferably for about 19.5 hours. Preferably, the slurry is heated while stirring. Preferably, the heating is to a temperature of about 60° C. to about 90° C. More preferably, the slurry is heated to about reflux temperature.

Preferably, the cooling is to a temperature of about 30° C. to about −10° C., more preferably, to about room temperature.

The precipitated crystalline ibandronate sodium Form Beta may be recovered from the slurry by any method known to one of ordinary skill in the art. Preferably, the crystalline ibandronate sodium Form Beta is recovered by collecting the precipitate of crystalline ibandronate sodium Form Beta from the slurry by filtration, washing the precipitate, and drying the precipitate. Preferably, the precipitate is washed with 1-propanol. Preferably, the precipitate is dried under vacuum with heating, preferably at a temperature of about 30° C. to about 60° C., and more preferably at about 50° C. Preferably, the drying is done under a pressure of about 20 to about 30 mbar. The precipitate may be dried for about 19 hours to about 25 hours.

The invention also encompasses a process for preparing a mixture of crystalline ibandronate sodium Form C and Form Alpha.

The invention encompasses a process for preparing a mixture of crystalline ibandronate sodium Form C and Form Alpha comprising suspending ibandronic acid Form S1 in n-butanol; heating the suspension (Slurry); adding a sodium salt to the suspension; and cooling the suspension to obtain a precipitate of the mixture of crystalline ibandronate sodium Form C and Form Alpha, wherein the sodium salt is selected from the group consisting of sodium tert butoxide and sodium acetate anhydrous.

Prior to the heating step, the suspension is typically stirred. Preferably, the suspension is heated to a temperature of about 80° C. to about 115° C., and more preferably to a temperature of about 114° C. The suspension is typically stirred while heating. Preferably, the suspension is stirred while heating for about 1 hour to about 5 hours prior to the cooling step.

Preferably, the suspension is cooled to about 30° C. to about 10° C., and more preferably to about room temperature. The suspension is typically stirred while cooling. Preferably, the suspension is stirred while cooling for about 16 hours to about 48 hours, and more preferably for about 24 hours to about 48 hours.

The precipitated mixture of crystalline ibandronate sodium Form C and Form Alpha may be recovered from the suspension by any method known to one of ordinary skill in the art. Preferably, the precipitated mixture of crystalline ibandronate sodium Form C and Form Alpha is recovered by collecting the precipitate from the suspension by filtration, washing the precipitate, and drying the precipitate. Preferably, the precipitate is washed with n-butanol. Preferably, the precipitate is dried under vacuum with heating, preferably at a temperature of about 30° C. to about 60° C., and more preferably at a temperature of about 50° C. Preferably, the drying is done under a pressure of about 10-200 mm Hg. The precipitate may be dried for about 19 hours to about 20 hours.

The invention also encompasses a crystalline form of ibandronate sodium denominated Form n-butanol solvate. Form n-butanol solvate is characterized by x-ray powder diffraction reflections at 9.2, 13.9, 16.6, and 18.5°2θ±0.2°2θ. Form n-butanol solvate can be further characterized by x-ray powder diffraction reflections at 4.6, 13.1, 17.2, 20.4, 23.2, and 24.5°2θ±0.2°2θ. FIG. 8 illustrates a representative powder x-ray diffraction diagram for Form n-butanol solvate. Form n-butanol solvate may have about 9 to about 15 wt % n-butanol, as determined by TGA. Form n-butanol solvate may be hemi or mono butanolate.

The crystalline ibandronate sodium Form n-butanol solvate may be prepared by a process comprising dissolving ibandronate sodium in water; combining the solution with n-butanol; and removing the water and n-butanol to obtain a precipitate of the crystalline ibandronate sodium Form n-butanol solvate.

The water may be heated to promote dissolution of the ibandronate sodium. Preferably, the water is heated to a temperature of about 50° C. to about 90° C., and more preferably to a temperature of about 80° C.

Preferably, the solution is combined with the n-butanol by adding the solution to the n-butanol.

Typically, the water and n-butanol are removed in the form of an azeotrope. Preferably, the azeotrope of water and n-butanol is removed by distillation, preferably at a temperature of about 80° C. to about 120° C., more preferably, to about 115° C. Preferably, the water and n-butanol are removed in an amount sufficient to induce precipitation of the crystalline ibandronate sodium Form n-butanol solvate, and more preferably about one-half of the volume of the water and n-butanol is removed.

Preferably, after removing the water and n-butanol, the solution is cooled to induce precipitation of the crystalline ibandronate sodium Form n-butanol solvate. Preferably, the solution is cooled to a temperature of about 30° C. to about 0° C., and more preferably to a temperature of about 25° C.

The precipitated crystalline ibandronate sodium Form n-butanol solvate may be recovered from the solution by any method known to one of ordinary skill in the art. Preferably, the crystalline ibandronate sodium Form n-butanol solvate is recovered by collecting the precipitate of crystalline ibandronate sodium Form n-butanol solvate from the solution by filtration, washing the precipitate, and drying the precipitate. Preferably, the precipitate is washed with diethyl ether.

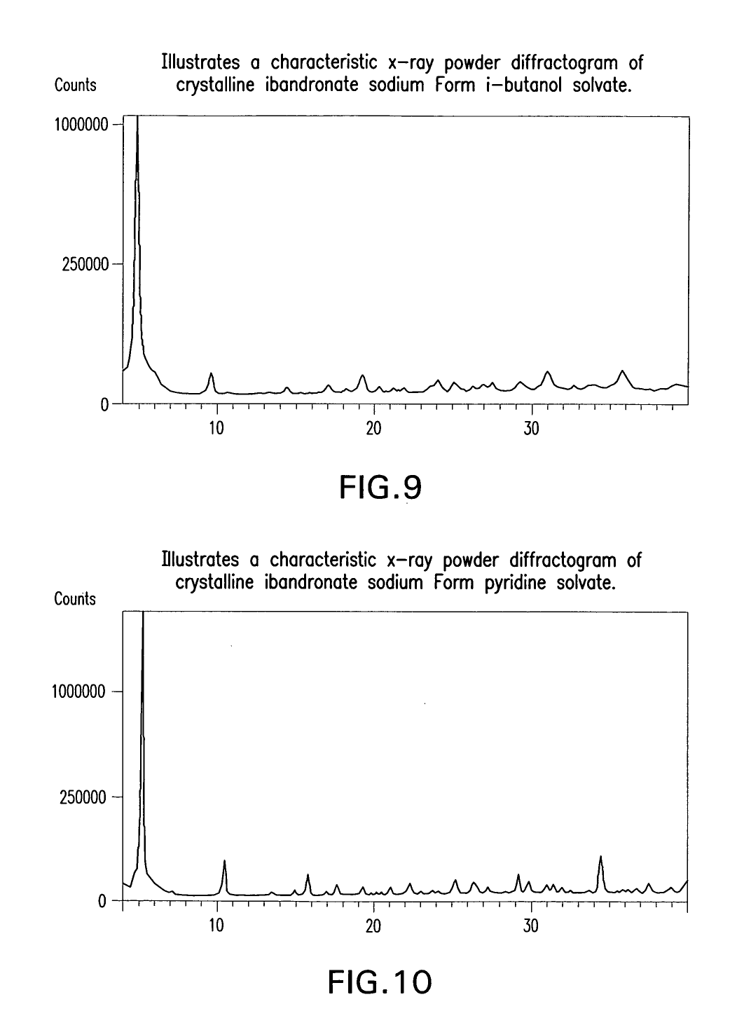

The invention also encompasses a crystalline form of ibandronate sodium denominated Form i-butanol solvate. Form i-butanol solvate is characterized by x-ray powder diffraction reflections at 9.6, 13.3, 14.4, and 19.3°2θ±0.2°2θ. Form i-butanol solvate can be further characterized by x-ray powder diffraction reflections at 4.8, 17.0, 18.2, 20.4, and 25.2°2θ±0.2°2θ. FIG. 9 illustrates a representative powder x-ray diffraction diagram for Form i-butanol solvate. Form i-butanol solvate may have about 15 to about 20 wt % i-butanol as determined by TGA. Form i-butanol solvate may be mono or sesquibutanolate.

The crystalline ibandronate sodium Form i-butanol solvate may be prepared by a process comprising dissolving ibandronate sodium in water; combining the solution with i-butanol; and removing the water and i-butanol to obtain a precipitate of the crystalline ibandronate sodium Form i-butanol solvate.

The water may be heated to promote dissolution of the ibandronate sodium. Preferably, the water is heated to a temperature of about 60° C. to about 100° C., and more preferably to a temperature of about 80° C.

Preferably, the solution is combined with the i-butanol by adding the solution to the i-butanol.

Typically, the water and i-butanol are removed in the form of an azeotrope. Preferably, the azeotrope of water and n-butanol is removed by distillation, preferably at a temperature of about 60° C. to about 110° C., more preferably, at about 98° C. Preferably, the water and i-butanol are removed in an amount sufficient to induce precipitation of the crystalline ibandronate sodium Form i-butanol solvate, and more preferably about one-half of the volume of the water and i-butanol is removed.

Preferably, after removing the water and i-butanol, the solution is cooled to induce precipitation of the crystalline ibandronate sodium Form i-butanol solvate. Preferably, the solution is cooled to a temperature of about 30° C. to about 0° C., and more preferably to a temperature of about 25° C.

The precipitated crystalline ibandronate sodium Form i-butanol solvate may be recovered from the solution by any method known to one of ordinary skill in the art. Preferably, the crystalline ibandronate sodium Form i-butanol solvate is recovered by collecting the precipitate of crystalline ibandronate sodium Form i-butanol solvate from the solution by filtration, washing the precipitate, and drying the precipitate. Preferably, the precipitate is washed with diethyl ether.

The invention also encompasses a crystalline form of ibandronate sodium denominated Form pyridine solvate. Form pyridine solvate is characterized by x-ray powder diffraction reflections at 10.6, 15.1, 15.9, and 17.8°2θ±0.2°2θ. Form pyridine solvate can be further characterized by x-ray powder diffraction reflections at 5.3, 19.5, 21.3, 22.5, and 25.4°2θ±0.2°2θ. FIG. 10 illustrates a representative powder x-ray diffraction diagram for Form pyridine solvate.

The crystalline ibandronate sodium Form pyridine solvate may be prepared by a process comprising dissolving ibandronate sodium in water; and adding pyridine to the solution to obtain a precipitate of the crystalline ibandronate sodium Form pyridine solvate.

The water may be heated to promote dissolution of the ibandronate sodium. Preferably, the water is heated to a temperature of about 60° C. to about 90° C., and more preferably to a temperature of about 80° C.

Preferably, the solution is cooled to induce precipitation of the crystalline ibandronate sodium Form pyridine solvate. Preferably, the solution is cooled to a temperature of about 30° C. to about 0°, and more preferably to a temperature of about 25° C.

The precipitated crystalline ibandronate sodium Form pyridine solvate may be recovered from the solution by any method known to one of ordinary skill in the art. Preferably, the crystalline ibandronate sodium Form pyridine solvate is recovered by collecting the precipitate of crystalline ibandronate sodium Form pyridine solvate from the solution by filtration, washing the precipitate, and drying the precipitate. Preferably, the precipitate is washed with diethyl ether.

The invention also encompasses a pharmaceutical formulation comprising at least one of the above-described crystalline ibandronate sodium Forms C2, Alpha, S4, S5, Beta, i-butanol solvate, or n-butanol solvate, and at least one pharmaceutically acceptable excipient.

The invention further encompasses a process for preparing a pharmaceutical formulation comprising combining at least one of the above-described crystalline ibandronate sodium Forms C2, Alpha, S4, S5, Beta, i-butanol solvate, or n-butanol solvate, with at least one pharmaceutically acceptable excipient.

The invention further encompasses the use of the above-described crystalline ibandronate sodium Forms C2, Alpha, S4, S5, Beta, i-butanol solvate, or n-butanol solvate, in the manufacture of a pharmaceutical composition.

Crystalline ibandronate sodium Forms Alpha, C, C2 and n-butanolate are stable at a temperature of about 15° C. to about 30° C. for more than about 3 months, as described in Example 17 below.

Crystalline ibandronate sodium Forms Alpha, C, C2 and n-butanolate have a purity of more than 90%, preferably, about 95%, most preferably, about 99%, of the crystalline ibandronate sodium in a given sample as measured by XRD. Preferably, the crystalline ibandronate sodium in a given sample is Form QQ.

The invention also encompasses crystalline ibandronate sodium Forms C2, Alpha, S4, S5, Beta, n-butanol solvate, i-butanol solvate, pyridine solvate, as defined in any of the embodiments mentioned herein, having a maximal particle size of less than or equal to about 500 μm, more preferably less than or equal to about 300 μm, even more preferably less than or equal to about 200 μm, even more preferably less than or equal to about 100 μm, and most preferably less than or equal to about 50 μm. As used herein, unless otherwise defined, the term “maximal particle size,” when used to described a sample of crystalline ibandronate sodium, means that 99% of the particles in the sample have a particle size of less than or equal to the maximal particle size. The particle size of the ibandronate sodium crystalline forms may be measured by methods such as sieves, sedimentation, electrozone sensing (coulter counter), microscopy, and/or Low Angle Laser Light Scattering (LALLS).

Pharmaceutical formulations of the invention contain crystalline ibandronate sodium, such as one of the above-described forms, and optionally one or more other forms of ibandronate sodium. In addition to the active ingredient, the pharmaceutical formulations of the invention can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. EUDRAGIT®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. CARBOPOL®), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the invention include maltol, vanillin, ethyl vanillin, menthol, citric acid, fumaric acid, ethyl maltol, and tartaric acid.

Solid compositions can also be dyed using any pharmaceutically acceptable colorant to improve their appearance and/or facilitate patient identification of the product and unit dosage level.

The solid compositions of the invention include powders, granulates, aggregates, and compacted compositions. The dosages include dosages suitable for oral, buccal, rectal, parenteral (including subcutaneous, intramuscular, and intravenous), inhalant, and ophthalmic administration. The dosages can be conveniently presented in unit dosage form and prepared by any of the methods well-known in the pharmaceutical arts.

Solid dosage forms include tablets, powders, capsules, suppositories, sachets, troches, and lozenges, as well as suspensions.

The dosage form of the invention can be a capsule containing the composition, preferably a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the invention can comprise any of the aforementioned blends and granulates that were described with reference to tableting, but they are not subjected to a final tableting step.

The invention also encompasses methods of treating or preventing skeletal-related events, such as osteoporosis, comprising administering a pharmaceutical formulation comprising a therapeutically effective amount of at least one of the above-described crystalline forms of ibandronate sodium and at least one pharmaceutically acceptable excipient to a patient in need thereof. Ibandronate sodium may be formulated for administration to a mammal, preferably a human, by injection. The crystalline ibandronate sodium can be formulated, for example, as a suspension for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. See, e.g., Ansel, H. C., et al., Pharmaceutical Dosage Forms and Drug Delivery Systems (7th ed. 1999), which is incorporated herein by reference.

Having thus described the invention with reference to particular preferred embodiments, those in the art can appreciate modifications to the invention as described and illustrated that do not depart from the spirit and scope of the invention as disclosed in the specification. The following examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Brittain, H. G., Polymorphism in Pharmaceutical Solids, Drugs and the Pharmaceutical Sciences, vol. 95 (Marcel Dekker, Inc. 1999) can be used for guidance. All references mentioned herein are incorporated in their entirety.

### EXAMPLES

### X-Ray Powder Diffraction

The x-ray powder diffraction was performed on Scintag X-ray powder diffractometer model X'TRA with a solid state detector at room temperature. Copper radiation of 1.5418 Å was used. The sample holder was a round standard aluminum sample holder with rough zero background. The scanning parameters were range: 2-40 degrees two-theta; scan mode: continuous scan; step size: 0.05 degrees; and at a rate of 5 degrees/minute.

In the examples below, room temperature is about 25° C.

### Thermal Gravimetric Analysis:

Typically to determine the Loss on Drying (LOD) by Thermal Gravimetric Analysis (TGA), a sample was heated from about 25° C. to about 250° C. at a heating rate of about 10° C. per minute, while purging with nitrogen gas at a flow rate of 40 ml/min.

### Example 1

### Process for Preparing Crystalline Ibandronate Sodium Form Alpha

Ibandronic Acid Form S1 (5.0 g) was stirred in abs ethanol (100 ml) at 78° C. To the slurry, sodium carbonate anhydrous (0.83 g, 1 eq) was added. The slurry was stirred at 78° C. for 2 hours and then it was cooled to room temperature. The slurry was stirred at room temperature for 16 hours. The precipitate was isolated by vacuum filtration, washed with abs ethanol (2×5 ml) and dried in a vacuum oven at 50° C. for 20 hours to obtain 5.84 g of crystalline ibandronate sodium Form Alpha.

### Example 2

### Process for Preparing Crystalline Ibandronate Sodium Form C2

Ibandronic Acid Form S1 (5.0 g) was stirred in n-butanol (100 ml) at 114° C. To the obtained partially solution, 30% sodium methoxide in methanol (2.82 g, 1 eq) was added. The slurry was stirred at 114° C. for 2.5 hours and then it was cooled to room temperature. The slurry was stirred at room temperature for 48 hours. The precipitate was isolated by vacuum filtration, washed with n-butanol (2×5 ml) and dried in a vacuum oven at 50° C. for 19.5 hours to obtain 4.14 g of ibandronate sodium crystal Form C2.

### Example 3

### Process for Preparing Crystalline Ibandronate Sodium Form Alpha

Ibandronic Acid Form S1 (5.0 g) was stirred in n-butanol (100 ml) at 114° C. To the obtained partial solution, sodium carbonate anhydrous (0.83 g, 1 eq) was added. The obtained slurry was stirred at 114° C. for 1 hour and then it was cooled to room temperature. The slurry was stirred at room temperature for 48 hours. The precipitate was isolated by vacuum filtration, washed with n-butanol (2×5 ml) and dried in a vacuum oven at 50° C. for 19.5 hours to obtain 5.77 g of ibandronate sodium crystal Form Alpha.

### Example 4

### Process for Preparing Crystalline Ibandronate Sodium Form S4

Ibandronic Acid Form S1 (5.0 g) was stirred in 1-propanol (100 ml) at 95° C. To the obtained partial solution, sodium hydrogen carbonate (1.3 g, 1 eq) was added. A slurry was obtained during the 2 hours stirring at 81° C. Then the slurry was cooled to room temperature. The slurry was stirred at room temperature for 16 hours. The precipitate was isolated by vacuum filtration, washed with 1-propanol (2×5 ml) and dried in a vacuum oven at 50° C. for 24 hours to obtain 6.53 g of crystalline ibandronate sodium Form S4.

### Example 5

### Process for Preparing Crystalline Ibandronate Sodium Form S5

Ibandronic Acid Form S1 (5.0 g) was stirred in 2-propanol (100 ml) at 81° C. to form a slurry. Sodium carbonate anhydrous (0.83 g, ½ eq) was then added to the slurry. The slurry was stirred at 81° C. for 2 hours and then it was cooled to room temperature. The slurry was stirred at room temperature for 16 hours. The precipitate was isolated by vacuum filtration, washed with 2-propanol (2×5 ml) and dried in a vacuum oven at 50° C. for 20 hours to obtain. 6.53 g of crystalline ibandronate sodium Form S5.

### Example 6

### Process for Preparing Crystalline Ibandronate Sodium Form Alpha

Ibandronic Acid Form S1 (5.0 g) was stirred in n-butanol (100 ml) at 114° C. To the obtained partial solution, sodium citrate dihydrate (1.54 g, 1 eq) was added. The slurry that was obtained after half an hour was stirred at 114° C. for 1.5 hours and then it was cooled to room temperature. The slurry was stirred at room temperature for 48 hours. The precipitate was isolated by vacuum filtration, washed with n-butanol (2×5 ml) and dried in a vacuum oven at 50° C. for 19.5 hours to obtain 6 g of crystalline ibandronate sodium Form Alpha.

### Example 7

### Process for Preparing Crystalline Ibandronate Sodium Form Alpha

Ibandronic Acid Form S1 (5.0 g) was stirred in n-butanol (100 ml) at 114° C. To the obtained partial solution, sodium hydrogen carbonate (1.3 g, 1 eq) was added. The slurry that was obtained stirred at 114° C. for 1 hour and then it was cooled to room temperature. The slurry was stirred at room temperature for 48 hours. The precipitate was isolated by vacuum filtration, washed with n-butanol (2×5 ml) and dried in a vacuum oven at 50° C. for 19.5 hours to obtain 6 g of crystalline ibandronate sodium Form Alpha.

### Example 8

### Process for Preparing Crystalline Ibandronate Sodium Forms C+Alpha

Ibandronic Acid Form S1 (5.0 g) was stirred in n-butanol (100 ml) at 114° C. To the obtained partial solution, sodium tert butoxide (0.8 g, 1 eq) was added. The slurry that was obtained after a period of time was stirred at 114° C. for 1 hour and then it was cooled to room temperature. The slurry was stirred at room temperature for 48 hours. The precipitate was isolated by vacuum filtration, washed with n-butanol (2×5 ml) and dried in a vacuum oven at 50° C. for 19.5 hours to obtain 6 g of crystalline ibandronate sodium Forms C+Alpha.

### Example 9

### Process for Preparing Crystalline Ibandronate Sodium Forms C+Alpha

Ibandronic Acid Form S1 (5.0 g) was stirred in n-butanol (100 ml) at 114° C. To the obtained partial solution, sodium acetate anhydrous (1.3 g, 1 eq) was added. The obtained slurry was stirred at 114° C. for 3 hours and then it was cooled to room temperature. The slurry was stirred at room temperature for 16 hours. The precipitate was isolated by vacuum filtration, washed with n-butanol (2×5 ml) and dried in a vacuum oven at 50° C. for 19.5 hours to obtain 5.7 g of crystalline ibandronate sodium Forms C+Alpha.

### Example 10

### Process for Preparing Crystalline Ibandronate Sodium Form Alpha

Ibandronic Acid Form S1 (5.0 g) was stirred in 2-butanol (100 ml) at 98° C. To the obtained partial solution, sodium citrate dihydrate (1.54 g, 1 eq) was added. The obtained slurry was stirred at 114° C. for 3 hours and then it was cooled to room temperature. The slurry was stirred at room temperature for 16 hours. The precipitate was isolated by vacuum filtration, washed with 2-butanol (2×5 ml) and dried in a vacuum oven at 50° C. for 19.5 hours to obtain 3.6 g of crystalline ibandronate sodium Form Alpha.

### Example 11

### Process for Preparing Crystalline Ibandronate Sodium Form Alpha

Ibandronic Acid Form S1 (5.0 g) was stirred in 2-butanol (100 ml) at 98° C. To the obtained partial solution, sodium hydrogen carbonate (1.3 g, 1 eq) was added. The obtained slurry was stirred at 114° C. for 3 hours and then it was cooled to room temperature. The slurry was stirred at room temperature for 16 hours. The precipitate was isolated by vacuum filtration, washed with 2-butanol (2×5 ml) and dried in a vacuum oven at 50° C. for 19.5 hours to obtain 5.66 g of crystalline ibandronate sodium Form Alpha.

### Example 12

### Process for Preparing Crystalline Ibandronate Sodium Form Beta

Ibandronic Acid (3.0 g, 1 eq) and IBD-Na₂ (3.4 g, 1 eq) were stirred in 1-Propanol (150 ml, 23.5 vol.) at reflux temperature for 19.5 hours. The slurry was cooled to room temperature and the precipitate was isolated by vacuum filtration, under nitrogen, washed with 1-Propanol (2×5 ml) and dried in a vacuum oven at 50° C. for 22.5 hours to obtain 6.92 g of Ibandronate Sodium Form Beta.

### Example 13

### Process for Preparing Crystalline Ibandronate Sodium Form C2

Ibandronate Sodium Form V (4.0 g) was stirred in abs Ethanol (120 ml) at room temperature. The slurry was stirred for 21 hours at room temperature. The precipitate was isolated by vacuum filtration, washed with abs Ethanol (2×5 ml) and dried in a vacuum oven at 50° C. for 24 hours to obtain 3.98 g of crystalline Ibandronate Sodium Form C2.

### Example 14

### Process for Preparing Crystalline Ibandronate Sodium n-butanol Solvate

Sodium ibandronate (300 mg) was dissolved in water at 80° C. (1 ml). The solution was gradually added to n-butanol (20 ml) heated to 115° C. About one half of volume of the resulting mixture was distilled off in the form of azeotrope of water and n-butanol. The resulting suspension was allowed to cool to 25° C. and left standing for an additional 3 hours, during which time sodium ibandronate crystallized from the solution. The resulting crystals of sodium ibandronate were filtered, washed with diethyl ether and dried by a nitrogen stream. Yield 290 mg.

### Example 15

### Process for Preparing Crystalline Ibandronate Sodium i-butanol Solvate

Sodium ibandronate (300 mg) was dissolved in water at 80° C. (1 ml). The solution was gradually added to i-butanol (20 ml) heated to 98° C. About one half of volume of the mixture was distilled off in the form of azeotrope of water and n-butanol. The resulting suspension was allowed to cool to 25° C. and left standing at 25° C. for an additional 3 hours, during which time sodium ibandronate crystallized from the solution. The resulting crystals of sodium ibandronate were filtered, washed with diethyl ether and dried by a nitrogen stream. Yield 310 mg.

### Example 16

### Process for Preparing Crystalline Ibandronate Sodium Pyridine Solvate

Sodium ibandronate (300 mg) was dissolved in water at 80° C. (0.8 ml). The solution was allowed to cool to 40° C. and pyridine (2 ml) was gradually added to the solution. The solution was then allowed to cool to 25° C. and left standing at 25° C. for an additional 5 hours, during which time sodium ibandronate crystallized from the solution. The resulting crystals of sodium ibandronate were filtered from the solution, washed with diethyl ether and dried by a nitrogen stream. Yield 290 mg.

### Example 17

### Stability of Crystalline Forms of Ibandronate Sodium when Stored for 3 Months at Room Temperature

0.5 g samples of crystalline ibandronate sodium Forms Alpha, C, C2 and n-butanolate, were each placed into polyethylene bags which were placed into Aluminium bags, which were then closed and stored at 25° C. for 3 months.

Table 1 summarizes the results before and after the storage.

| | |
|---|---|
| Time Interval | Results |

| Temperature | not applicable | 25° C. |
|---|---|---|
| T = 0 | Form Alpha | |
| 1 month | — | Alpha |
| 3 months | — | Alpha |
| T = 0 | Form C | |
| 1 month | — | C |
| 3 months | — | C |
| T = 0 | Form C2 | |
| 1 month | — | C2 |
| 3 months | — | C2 |
| T = 0 | Form n-butanolate | |
| 1 month | — | n-butanolate |
| 3 months | — | n-butanolate |

As illustrated by Table 2 above, Forms Alpha, C, C2 and n-butanolate maintained their crystalline form after storage at room temperature for 3 months.

### Example 18

### Process for Preparing Crystalline Ibandronate Disodium Form DS2

Ibandronic acid (50 g) was dissolved in water (333.5 ml, 6.67 vol.) at room temperature. The solution was heated to reflux (70° C.). NaOH(s) (12.53 g, 2 eq) was added to the solution. Acetone (500 ml) was added drop-wise to the solution over 7 minutes at 92° C. The solution was stirred at reflux for 12 minutes and then cooled to room temperature. The solution was then stirred at room temperature for 80.5 hours to form a slurry. The precipitated was isolated from the slurry by vacuum filtration under nitrogen, washed with acetone (2×50 ml) and dried in a vacuum oven at 50° C. for 22.5 hours to give 45 g of ibandronate disodium Form DS2.

### Example 19

### Process for Preparing Crystalline Ibandronate Sodium Form C

Ibandronic Acid Form S1 (5.0 g) was stirred in n-butanol (100 ml) at 114° C. To the obtained partial solution, sodium tetraborate decahydrate (3 g, 1 eq) was added. The obtained slurry was stirred at 114° C. for 3 hours and then it was cooled to room temperature. The slurry was stirred at room temperature for 16 hours. The precipitate was isolated by vacuum filtration, washed with n-butanol (2×5 ml) and dried in a vacuum oven at 50° C. for 19.5 hours to obtain 6.2 g of crystalline ibandronate sodium Form C.

### Example 20

### Process for Preparing Crystalline Ibandronate Sodium Form V

Crystalline ibandronate sodium Form C was stored at room temperature for 2 years. The sample was retested after the storage time and found to be crystalline ibandronate sodium Form V by XRD.

1-8. (canceled)

9. A crystalline form of ibandronate sodium characterized by x-ray powder diffraction reflections at 11.8, 16.2, 17.8, and 18.8°2θ±0.2°2θ.

10. The crystalline form of ibandronate sodium of claim 9, further characterized by x-ray powder diffraction reflections at 10.2 and 20.0°2θ±0.2°2θ.

11. The crystalline form of ibandronate sodium of claim 9, further characterized by a powder x-ray diffraction pattern as depicted in FIG. 2 or 3.

12. The crystalline form of ibandronate sodium of claim 9, wherein the crystalline form is monohydrate.

13. A process for preparing a crystalline form of ibandronate sodium characterized by x-ray powder diffraction reflections at 11.8, 16.2, 17.8, and 18.8°2θ±0.2°2θ comprising suspending crystalline ibandronic acid characterized by x-ray powder diffraction reflections at 8.2, 11.5, 11.9, 13.9, 18.6 and 22.2°2θ±0.2°2θ with a solvent; heating the suspension; adding a sodium salt to the suspension to obtain ibandronate sodium; and cooling the suspension to obtain a precipitate of the crystalline form of ibandronate sodium, wherein the solvent is selected from the group consisting of ethanol, n-butanol, and 2-butanol, and the sodium salt is selected from the group consisting of sodium carbonate anhydrous, sodium citrate dihydrate and sodium hydrogen carbonate.

14. The process of claim 13, wherein the suspension is heated to a temperature of about 78° C. to about 114° C.

15. The process of claim 13, wherein the suspension is cooled to about 30° C. to about 10° C.

16-60. (canceled)

61. A pharmaceutical formulation comprising the crystalline form of ibandronate sodium of claim 9
and at least one pharmaceutically acceptable excipient.

62. A process for preparing a pharmaceutical formulation comprising combining the crystalline form of ibandronate sodium of claim 9
with at least one pharmaceutically acceptable excipient.

63. A method of treating or preventing skeletal-related events comprising administering a pharmaceutical formulation comprising a therapeutically effective amount of the crystalline form of ibandronate sodium of claim 9
and at least one pharmaceutically acceptable excipient to a patient in need thereof.

64. The method of claim 63, wherein the skeletal-related event is osteoporosis.
